(19) United States
(12) Patent Application Publication
(10) Pub. No.: US 2025/0042787 A1
(43) Pub. Date: Feb. 6, 2025

(54) BIOGENIC MANGANESE OXIDE (BMO)@SPONGE BIOMATERIAL, AND PREPARATION METHOD AND APPLICATION THEREOF

(71) Applicant: QINGDAO UNIVERSITY OF TECHNOLOGY, Qingdao (CN)

(72) Inventors: Huawei WANG, Qingdao (CN); Xueqin WANG, Qingdao (CN); Rongxue ZOU, Qingdao (CN); Xiqing CUI, Qingdao (CN); Mengfan LIU, Qingdao (CN); Yanan WANG, Qingdao (CN); Zhibo YANG, Qingdao (CN); Shupeng LI, Qingdao (CN); Yingjie SUN, Qingdao (CN); Yujun WANG, Qingdao (CN)

(21) Appl. No.: 18/920,838

(22) Filed: Oct. 18, 2024

(30) Foreign Application Priority Data
Jun. 14, 2024 (CN) 202410765947.5

(51) Int. Cl. C02F 1/72; C02F 3/34; C02F 101/34; C12N 1/20

(52) U.S. Cl. CPC C02F 1/725; C02F 3/348; C12N 1/20; C02F 2101/34; C02F 2305/023

(57) ABSTRACT

A method for preparing a biogenic manganese oxide (BMO)@sponge biomaterial, in which a divalent manganese ion solution and a sponge carrier are added to a pre-sterilized culture medium, and then a manganese oxidizing bacterium with an accession number of ATCC 23483 is inoculated. The culture medium was cultured and freeze-dried to obtain the BMO@sponge biomaterial. This application further provides a method for treating an antibiotic-containing wastewater, in which a BMO@sponge biomaterial prepared by such method and a peroxymonosulfate (PMS) added to the wastewater. The PMS is activated by the BMO@sponge biomaterial to generate active substances such as hydroxyl radicals, sulfate radicals and singlet oxygen radicals, which can efficiently degrade the antibiotic into CO₂ and water through a series of physical and chemical reactions and free radical chain reactions.

### CROSS-REFERENCE TO RELATED APPLICATIONS

This application claims the benefit of priority from Chinese Patent Application No. 202410765947.5, filed on Jun. 14, 2024. The content of the aforementioned application, including any intervening amendments made thereto, is incorporated herein by reference in its entirety.

### TECHNICAL FIELD

This application relates to water environment restoration and treatment, and more particularly to a biogenic manganese oxide (BMO)@sponge biomaterial, and a preparation method and application thereof.

### BACKGROUND

The rapid development of the pharmaceutical industry has greatly expanded the application of antibiotic-type antibacterial or bactericidal drugs. In China, the total consumption of veterinary antibiotics reached 33,000 t in 2020. Tetracyclines are one of the most widely used antibiotics in clinical and agricultural applications. The parent compounds of antibiotics are highly stable, and cannot be fully absorbed and utilized by human and animal bodies. Most antibiotics eventually enter the water environment and cause pollution. Some antibiotics, such as tetracycline and sulfadiazine, have been frequently detected in water bodies such as surface water, groundwater and drinking water, even reaching the mg/L levels. High concentrations of antibiotics are not only toxic to organisms, but also lead to increased drug resistance in sensitive strains and wide spread of antibiotic resistance genes. Therefore, it is of great significance to develop technologies for efficient degradation of tetracyclines.

The existing methods for treating antibiotic wastewater include biological, physical, and chemical methods. Advanced oxidation processes (AOPs) based on free radicals such as hydroxyl radicals (.OH) or sulfate radicals (SO₄·⁻) can effectively degrade the antibiotics. Compared to hydroxyl radicals, sulfate radicals have longer half-lives, stronger oxidizing properties, and wider pH range (pH 3-9), and can degrade the organic pollutants more thoroughly. Sulfate radicals can be generated by the activation of peroxymonosulfate (PMS). The activation of PMS can be achieved by means of transition metal oxides, carbon materials, heating, ultraviolet light, etc. It has been shown that metal oxides such as iron, manganese, and cobalt can be adopted to activate PMS, thereby degrading antibiotics such as tetracycline, ofloxacin, and norfloxacin. However, the synthesis of traditional metal oxides is a complex process, which commonly requires dangerous and expensive compounds as reducing agents or stabilizers, as well as extreme conditions such as high temperature and high pressure, thereby limiting their application scope. In addition, the dissolution of metal oxides may cause the release of metal ions, which are difficult to recover and easily cause secondary pollution.

In view of the shortcomings of traditional chemical synthesis of metal oxides, biosynthesis can be used as one of the important alternatives to metal oxide catalysts. Many microorganisms (such as manganese oxidizing bacteria (MnOB)) can induce the generation of biological manganese oxides (BMOs) through biosynthesis under neutral, normal temperature, and normal pressure conditions. Compared with chemically synthesized metal Mn oxides, BMO produced by manganese oxidizing bacteria has the characteristics of weak crystallinity, high Mn valence, and multiple holes in its octahedral structure, resulting in a stronger reaction activity.

It has been shown that BMO can activate PMS or induce the generation of free radicals in a Fenton-like manner, thereby accelerating the degradation of organic pollutants. For example, biogenic MnO₂ can activate PMS to produce singlet oxygen through self-decomposition and energy quenching mechanisms, which can effectively degrade organic matter. Tian et al. (Tian. N., Tian, X., Nie, Y., Yang, C., Zhou, Z., Li, Y., 2018. Biogenic manganese oxide: An efficient peroxymonosulfate activation catalyst for tetracycline and phenol degradation in water. Chemical Engineering Journal, 352:469-476) have shown that BMO has unprecedented efficiency and stability in the degradation of phenol and tetracycline by PMS activation, the reaction rate of which is three times higher than that of chemically synthesized manganese oxides. The bio-iron manganese oxide (bio-FeMnOₓ) prepared by Du et al. (Du, Z., Li, K., Zhou, S., Liu, X., Yu, Y., Zhang, Y., He, Y., Zhang, Y., 2020. Degradation of ofloxacin with heterogeneous photo-Fenton catalyzed by biogenic Fe-Mn oxides. Chemical Engineering Journal, 380:122427) was applied to the heterogeneous photo-Fenton method to catalyze the degradation of moxifloxacin, and with a catalytic activity of 2 times higher than that of chemically synthesized iron manganese oxide. Thongpitak et al. (Thongpitak, J., Pumas, P., Pumas, C., 2020. Paraquat degradation by biological manganese oxide (BioMnOₓ) catalyst generated from living microalga Pediastrum duplex AARL G060. Frontiers in Microbiology, 11:575361) used BioMnOₓ as a catalyst for the Fenton-like reaction to remove pollutants from real wastewater. However, it has been shown that when the synthesized BMO is directly added to the reactor for use, disadvantages such as easy agglomeration of BMO, poor mass transfer effect and limited active sites will occur, resulting in a poor pollutant removal effect.

Therefore, there is an urgent need for an antibiotic wastewater treatment method with excellent antibiotic degradation effect, low cost and no secondary pollution.

### SUMMARY

An object of the disclosure is to provide a biogenic manganese oxide (BMO)@sponge biomaterial, and a preparation method and application thereof, which has simple process, short treatment time, convenient operation, low treatment cost, wide treatment range and no secondary pollution, and can effectively degrade the antibiotics in wastewater.

In order to achieve the above object, the following technical solutions are adopted.

In a first aspect, this application provides a BMO@sponge biomaterial, wherein the BMO@sponge biomaterial is prepared by loading a manganese oxidizing bacterium and a divalent manganese ion on a sponge carrier.

In some embodiments, the manganese oxidizing bacterium is MnB 1 deposited at American Type Culture Collection (ATCC) with an accession number of ATCC 23483.

In a second aspect, this application provides a method for preparing the above BMO@sponge biomaterial, comprising:
preparing and sterilizing a culture medium; sequentially adding a divalent manganese ion stock solution and the sponge carrier to the culture medium; and
inoculating the manganese oxidizing bacterium into the culture medium followed by incubation and freeze-drying to obtain the BMO@sponge biomaterial.

In some embodiments, the culture medium comprises 0.15 g/L ammonium ferrous sulfate, 0.075 g/L yeast leaching powder, 0.15 g/L sodium citrate, 0.05 g/L sodium pyrophosphate and 20 mM N-2-hydroxyethylpiperazine-N-2-ethanesulfonic acid, and has a pH of 7.0-7.2.

In some embodiments, the divalent manganese ion stock solution is a MnSO₄·7H₂O solution, and the MnSO₄·7H₂O solution is added such that a final divalent manganese ion concentration in the culture medium is 5-20 mg/L.

In some embodiments, the sponge carrier has a size of 1×1×1 cm, and an addition amount of the sponge carrier is 20-40 blocks/L.

In some embodiments, the manganese oxidizing bacterium is cultured to a logarithmic phase with an optical density at 600 nm (OD₆₀₀) of 0.9-1.2, and inoculated into the culture medium at an inoculum amount of 1%-5% by volume of the culture medium.

In a third aspect, this application provides a method for treating a wastewater polluted by an antibiotic, comprising:
adding the above BMO@sponge biomaterial and peroxymonosulfate (PMS) to the wastewater to degrade the antibiotic.

In some embodiments, the antibiotic is selected from the group consisting of tetracycline, macrolide, aminoglycoside and a combination thereof.

In some embodiments, a dosage of the BMO@sponge biomaterial is 5-15 blocks/L with each block having a size of 1 cm×1 cm×1 cm; a dosage of the PMS is 50-400 mg/L; and the wastewater has a pH of 7-9.

Compared to the prior art, the present disclosure has the following beneficial effects.
(1) The BMO@sponge biomaterial provided herein is prepared through microbial synthesis instead of chemical synthesis, thereby avoiding excessive consumption of chemical agents. Moreover, the whole process is performed under neutral and mild conditions.
(2) The BMO@sponge biomaterial of the present disclosure is a typical porous material, exhibiting a typical fibrous three-dimensional network structure with well-developed voids, excellent mass transfer effect, abundant active sites, diversified free radicals and strong activity.
(3) The BMO@sponge biomaterial of the present disclosure is a novel PMS catalytic activator with excellent stability and high efficiency, and has promising application prospects.
(4) The BMO@sponge biomaterial prepared herein can activate the PMS to degrade the antibiotic in the wastewater. Specifically, in the BMO@sponge and PMS system, PMS is activated by the BMO@sponge to generate a series of active substances such as a hydroxyl radical, a sulfate radical and a singlet oxygen radical, which further trigger a series of physical and chemical reactions and free radical chain reactions to degrade the antibiotic in the wastewater into CO₂ and water, thereby achieving the efficient removal of antibiotic pollutants. Moreover, this treatment method has simple process, less time consumption, convenient operation, low cost, wide treatment range and no secondary pollution. Regarding the tetracycline-polluted wastewater, the treatment method can reach a removal rate of 91.84%, the method of activating PMS by the BMO@sponge to degrade the antibiotic is adopted to treat, so that a tetracycline efficiency can reach. After repeatedly used 6 times, the BMO@sponge biomaterial still exhibits a desirable degradation efficiency for the antibiotic (78.7%).

### BRIEF DESCRIPTION OF THE DRAWINGS

In order to make the technical solutions of the embodiments of the present disclosure or the technical solutions in the prior art clearer, the accompanying drawings required in the description of the embodiments or prior art will be briefly described below. Obviously, presented in the drawings are merely some embodiments of the disclosure. For those of ordinary skill in the art, other drawings can be obtained based on the drawings of the disclosure without making creative efforts.

FIG. 1 is a scanning electron microscopy (SEM) image of a biogenic manganese oxide (BMO)@sponge biomaterial in Example 1 of the present disclosure;

FIG. 2 is an energy dispersive spectroscopy (EDS) spectrum of the BMO@sponge biomaterial in Example 1 of the present disclosure;

FIG. 3 is an X-ray diffraction (XRD) pattern of a BMO@sponge biomaterial in Example 2 of the present disclosure;

FIG. 4 shows tetracycline degradation efficiency of different reaction systems in Experimental Example 1 of the present disclosure;

FIGS. 5a-b are electron paramagnetic resonance (EPR) spectra of a BMO@sponge/PMS system in Experimental Example 6 of the present disclosure;

FIG. 6 shows change of tetracycline degradation efficiency of the BMO@sponge/PMS system with the number of cycles in Experimental Example 6 of the present disclosure; and

FIG. 7 shows released Mn²⁺ (Mn(II)) concentration during different cycles of the BMO@sponge/PMS system in Experimental Example 6 of the present disclosure.

### DETAILED DESCRIPTION OF EMBODIMENTS

The embodiments of the present disclosure will be described in detail below. The embodiments described herein are merely illustrative of the present application, and are not intended to limit the disclosure. Unless otherwise specified, techniques or conditions can be performed in accordance with those described in the literature in the art or product instructions, and the reagents or instruments are commercially available.

A biogenic manganese oxide (BMO)@sponge biomaterial is prepared through the following steps.

A culture medium is prepared and sterilized, and sequentially added with a divalent manganese ion stock solution and a cleaned sponge carrier. A manganese oxidizing bacterium is inoculated into the culture medium followed by incubation and freeze-drying, so as to obtain the BMO@sponge biomaterial.

The BMO@sponge biomaterial can activate peroxymonosulfate (PMS) to induce the generation of active substances such as a hydroxyl radical, a sulfate radical and a singlet oxygen radical, which further trigger cause a series of physical and chemical reactions and free radical chain reactions to degrade the antibiotic in the wastewater into CO₂ and water, thereby achieving the efficient removal of antibiotic pollutants.

The culture medium used herein includes 0.15 g/L ammonium ferrous sulfate, 0.075 g/L yeast leaching powder, 0.15 g/L sodium citrate, 0.05 g/L sodium pyrophosphate and 20 mM N-2-hydroxyethylpiperazine-N-2-ethanesulfonic acid (HEPES), and has a pH of 7.0.

The manganese oxidizing bacterium used herein is Pseudomonas putida strain MnB 1 deposited in American Type Culture Collection (ATCC) with an accession number of ATCC 23483.

### Example 1

Provided herein was a method for preparing a BMO@sponge biomaterial.

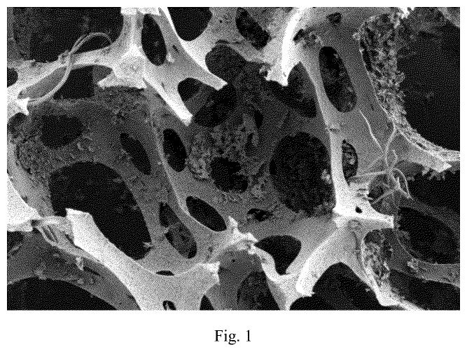

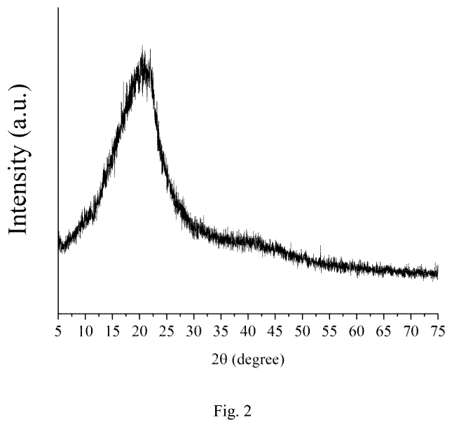

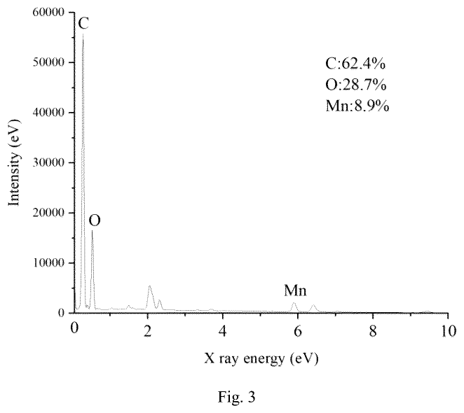

A culture medium was autoclaved at 121° C. for 15 min, and then was added with a MnSO₄·7H₂O solution to a concentration of 15 mg/L. A cleaned sponge carrier with a size of 1 cm (length)×1 cm (width)×1 cm (height) was added to the culture medium at an amount of 30 blocks/L. A MnB1 strain was inoculated into the culture medium at an amount of 5% by volume of the culture medium. The culture medium was cultured in an incubator at 120 rpm and 30° C. for 3 days. The sponge material was collected, washed with deionized water and 0.01 mol/L phosphate buffered saline (PBS) (pH=7.0) 5 times, and then freeze-dried at −60° C. to obtain the BMO@sponge biomaterial. The BMO@sponge biomaterial was characterized by scanning electron microscopy (SEM) and energy dispersive spectroscopy (EDS), and the results were shown in FIGS. 1 and 2.

Referring to the SEM image in FIG. 1, the sponge was a typical porous material. After the BMO was loaded, a network structure of the sponge was not blocked, but a rough and porous BMO mineral structure was presented on a fiber surface. It can be seen from the EDS spectrum in FIG. 2 that relative contents of C, O and Mn elements of the BMO@sponge biomaterial were 62.4%, 8.9% and 28.7%, respectively, indicating that BMO was successfully loaded on the surface of the sponge carrier.

### Example 2

Provided herein was a method for preparing a BMO@sponge biomaterial.

A culture medium was autoclaved at 121° C. for 15 min, and then was added with a MnSO₄·7H₂O solution to a concentration to 10 mg/L. A cleaned sponge carrier with a size of 1 cm (length)×1 cm (width)×1 cm (height) was added to the culture medium at an amount of 25 blocks/L. A MnB1 strain was inoculated into the culture medium at an amount of 3% by volume of the culture medium. The culture medium was cultured in an incubator at 120 rpm and 30° C. for 2 days. The sponge material was collected, washed with deionized water and 0.01 mol/L PBS (pH=7.0) 5 times, and then freeze-dried at −60° C., so as to obtain the BMO@sponge biomaterial. The BMO@sponge biomaterial was characterized, and the result was shown in FIG. 3.

Referring to the X-ray diffraction (XRD) pattern in FIG. 3, two diffraction peaks with weak intensity appeared near 20 of 20°, which were broad and blunt, indicating that the biosynthesized BMO was mainly weakly crystalline or amorphous. Therefore, the BMO enriched on the surface of the sponge carrier was a weakly crystalline or amorphous layered polymer manganese oxide.

### Example 3

Provided herein was a method for preparing a BMO@sponge biomaterial.

A culture medium was autoclaved at 121° C. for 15 min, and then was added with a MnSO₄·7H₂O solution to a concentration to 20 mg/L. A cleaned sponge carrier with a size of 1 cm (length)×1 cm (width)×1 cm (height) was added to the culture medium at an amount of 20 blocks/L. A MnB1 strain was inoculated into the culture medium at an amount of 3% by volume of the culture medium. The culture medium was cultured in an incubator at 120 rpm and 30° C. for 4 days. The sponge material was collected, washed with deionized water and 0.01 mol/L PBS (pH=7.0) 5 times, and then freeze-dried at −60° C., so as to obtain the BMO@sponge biomaterial.

### Experimental Example 1

The oxidative degradation effect of the BMO@sponge biomaterial (prepared in Example 1)-PMS on tetracycline was verified herein.

1 L of a wastewater sample containing 15 mg/L of tetracycline was adjusted to pH 9 with a 0.1 mol/L sodium hydroxide solution, and sequentially added with PMS with a concentration of 200 mg/L and the BMO@sponge biomaterial with a dosage of 10 blocks/L. The wastewater sample was regularly monitored for a change in pH value and maintained at pH 9, and sampled at regular intervals to monitor a change in the tetracycline concentration. The results were shown in Table 1.

TABLE 1
Degradation effect of BMO@sponge/PMS on the
tetracycline in wastewater at different reaction times

| | Reaction time (min) | | | | | | |
|---|---|---|---|---|---|---|---|
| | 10 | 20 | 30 | 45 | 60 | 120 | 180 |
| Degradation | 68.1 | 74.9 | 78.2 | 79.3 | 83.2 | 88.5 | 93.2 |
| efficiency (%) | | | | | | | |

As shown in Table 1, the use of BMO@sponge to activate PMS to degrade tetracycline exhibited an excellent catalytic activity. The degradation efficiency for tetracycline was 68.1% at 10 min, and increased to 93.2% at 180 min, indicating that the BMO@sponge can effectively activate PMS to induce the generation of free radicals such as sulfate radicals and hydroxyl radicals, thereby accelerating the oxidative degradation of tetracycline.

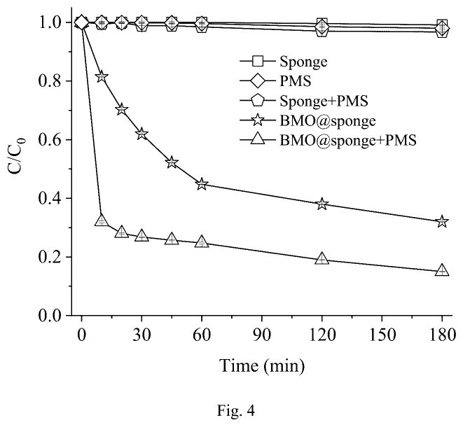

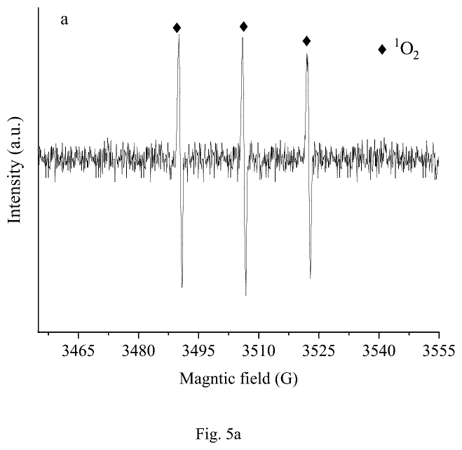

The above method was adopted, and blank sponge, PMS, blank sponge+PMS, and BMO@sponge were used as controls to detect effects of using different reaction systems to activate PMS on the degradation of tetracycline. A result was shown in FIG. 4.

As shown in FIG. 4, the BMO@sponge/PMS system exhibited the highest degradation efficiency for tetracycline, followed by the BMO@sponge system alone. This indicated that the BMO@sponge biomaterial can effectively activate PMS and achieve efficient degradation for tetracycline.

### Experimental Example 2

Effect of dosage of the BMO@sponge biomaterial prepared in Example 1 on the degradation of tetracycline was investigated.

1 L of a wastewater sample containing 15 mg/L of tetracycline was adjusted to pH 9 with a 0.1 mol/L sodium hydroxide solution, and sequentially added with PMS with a concentration of 200 mg/L and the BMO@sponge biomaterial with a dosage of 5, 8, 10, 12 and 15 blocks/L, respectively. The wastewater sample was regularly monitored for a change in pH value and maintained at pH 9. After reacting for 3 h, the effect of dosage of the BMO@sponge/PMS on the degradation of tetracycline in wastewater was tested. The results were shown in Table 2.

TABLE 2
Effect of dosage of BMO@sponge/PMS on the degradation of
tetracycline in wastewater

| BMO@sponge dosage | | | | | |
|---|---|---|---|---|---|
| (block/L) | 5 | 8 | 10 | 12 | 15 |
| Degradation efficiency (%) | 88.6 | 91.4 | 92.5 | 92.8 | 93.3 |

As shown in Table 2, the use of BMO@sponge at different dosages to activate PMS to degrade tetracycline exhibited an excellent catalytic activity.

The degradation efficiency for tetracycline was 88.6% at 5 blocks/L, and increased to 92.8% at 12 blocks/L.

### Experimental Example 3

Effect of concentration of the tetracycline in the wastewater on the degradation of tetracycline by the BMO@sponge biomaterial prepared in Example 2 was investigated.

1 L of wastewater samples containing 5, 10, 15, 20 and 25 mg/L of tetracycline, respectively, were each adjusted to pH 9 with a 0.1 mol/L sodium hydroxide solution, and sequentially added with PMS with a concentration of 200 mg/L and the BMO@sponge biomaterial with a dosage of 10 blocks/L. The wastewater sample was regularly monitored for the pH change and maintained at pH 9. After reacted for 3 h, the degradation effect of the BMO@sponge/PMS on different concentrations of tetracycline in the wastewater was analyzed. The results were shown in Table 3.

TABLE 3
Degradation effect of BMO@sponge/PMS on different
concentrations of tetracycline in wastewater

| Tetracycline concentration | | | | | |
|---|---|---|---|---|---|
| (g/L) | 5 | 10 | 15 | 20 | 25 |
| Degradation efficiency (%) | 96.5 | 94.8 | 93.9 | 91.6 | 89.2 |

As shown in Table 3, under different tetracycline concentrations, the PMS could be activated by the BMO@sponge to catalytically degrade the tetracycline in the wastewater. The degradation efficiency reached 96.5% at a tetracycline concentration of 5 mg/L, and decreased to 89.2% when the tetracycline concentration increased to 25 mg/L.

### Experimental Example 4

Degradation efficiency of the BMO@sponge biomaterial prepared in Example 3 against the tetracycline under different pH conditions was investigated.

1 L of wastewater samples containing 15 mg/L were adjusted to pH 3, 5, 7 and 9 with a 0.1 mol/L sodium hydroxide solution or a 0.1 mol/L hydrochloric acid solution, respectively. The wastewater samples were each sequentially added with PMS with a concentration of 200 mg/L and the BMO@sponge biomaterial with a dosage of 15 blocks/L. The wastewater samples were each regularly monitored for a change in pH value and maintained at pH 3, 5, 7 and 9, respectively. After reacting for 3 h, the effect of pH of wastewater on the degradation of tetracycline by the BMO@sponge/PMS was tested. The results were shown in Table 4.

TABLE 4
Effect of pH on the degradation efficiency of the BMO@sponge/PMS
against tetracycline

| pH value | 3 | 5 | 7 | 9 |
|---|---|---|---|---|
| Degradation efficiency (%) | 71.1 | 74.3 | 82.0 | 93.4 |

As shown in Table 4, the pH of the wastewater samples had a certain effect on the degradation efficiency of the BMO@sponge/PMS against tetracycline. The degradation efficiency for tetracycline was 82.0% at pH 7, and was 93.4% at pH 9. Under a strong alkaline condition (pH=9), the BMO@sponge biomaterial exhibited a higher catalytic degradation efficiency for tetracycline. This was because that under an alkaline condition, OH-can promote the decomposition of HSO₅⁻, thereby accelerating the generation of SO₄·⁻ free radicals. In addition, the strong alkaline condition can inhibit the dissolution of the Mn element.

### Experimental Example 5

Effect of PMS concentration on the degradation of tetracycline in the presence of the BMO@sponge biomaterial prepared in Example 3 was investigated.

1 L of wastewater samples containing 15 mg/L of tetracycline were each adjusted to pH 9 with a 0.1 mol/L sodium hydroxide solution, and sequentially added with PMS and a BMO@sponge material with a dosage of 15 blocks/L. The PMS concentrations were 50, 100, 200 and 400 mg/L, respectively. The wastewater sample was regularly monitored for a change in pH value and maintained at pH 9. After reacting for 3 h, the effect of PMS concentration on the degradation of tetracycline by the BMO@sponge/PMS was tested. The results were shown in Table 5.

TABLE 5
Effect of PMS concentration on the degradation of tetracycline by
the BMO@sponge/PMS

| PMS concentration (mg/L) | 50 | 100 | 200 | 400 |
|---|---|---|---|---|
| Degradation efficiency (%) | 67.4 | 75.6 | 93.7 | 84.5 |

As shown in Table 5, the PMS concentration had a certain effect on the degradation of tetracycline by using the BMO@sponge biomaterial to activate the PMS. The degradation efficiency for tetracycline was merely 67.4% at a PMS concentration of 50 mg/L, and increased to 93.7% a PMS concentration of 200 mg/L. This indicated that PMS can be effectively activated by the BMO@sponge biomaterial, thereby generating more ·OH and SO₄·⁻ radicals, so as to improve the degradation efficiency for tetracycline. However, excessive concentration of PMS can cause a sharp drop in degradation efficiency for tetracycline (only 84.5%).

### Experimental Example 6

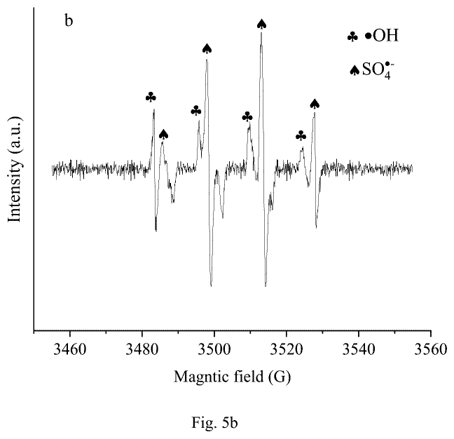

The reaction between the BMO@sponge biomaterial and PMS was further investigated as follows.
(1) 2,2,6,6-tetramethylpiperidine (TEMP) and 5,5-dimethyl-1-pyrroline N-oxide (DMPO) were used as traps. Signal characteristics of each active oxygen species were analyzed by electron paramagnetic resonance (EPR) to identify main types of free radicals in the BMO@sponge/PMS reaction system. The results were shown in FIGS. 5a-b.

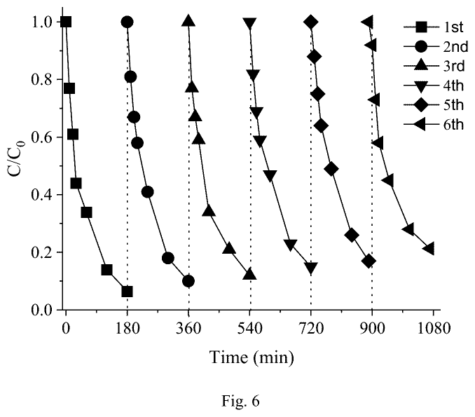

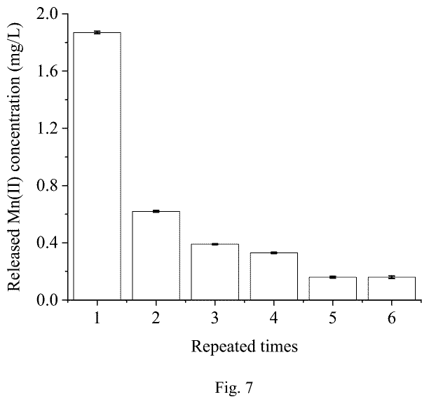

As shown in FIGS. 5a-b, obvious signal characteristic peaks of ·OH, SO₄·⁻ and ¹O₂ free radicals appeared in the reaction system, which indicated that these three free radicals play a vital role in the degradation process of tetracycline.
(2) The method of Example 1 was adopted, and the BMO@sponge biomaterial used to activate PMS was reused under the same conditions to test the degradation efficiency for tetracycline and the dissolution of the manganese ion under different reuse times. The results were shown in FIGS. 6 and 7.

As shown in FIG. 6, after repeatedly used 6 times, the degradation efficiency for tetracycline decreased from 93.5% to 78.7%. The main reasons for the decrease in the degradation efficiency for tetracycline are: 1) the consumption and dissolution release of BMO on the sponge surface with increasing repeated use times; and 2) tetracycline molecules occupy effective active sites on the BMO surface. As shown in FIG. 7, during the repeatedly using of the BMO@sponge biomaterial, the release of the Mn²⁺ ions occurred. As the repeated use times increased, the release of Mn²⁺ (Mn(II)) ions gradually decreased. The Mn²⁺ concentration was 1.87 mg/L after repeatedly used 1 time, and decreased to 0.16 mg/L after repeatedly used 6 times. In summary, the biosynthesized BMO@sponge biomaterial has excellent stability and reusability for repeated use.

The embodiments described above are merely illustrative of the present disclosure, and are not intended to limit the patent scope of the present in disclosure. It should be understood that any improvements and modifications made by those skilled in the art without departing from the spirit of the disclosure shall fall within the scope of the disclosure defined by the appended claims.

What is claimed is:

1. A biogenic manganese oxide (BMO)@sponge biomaterial, wherein the BMO@sponge biomaterial is prepared by loading a manganese oxidizing bacterium and a divalent manganese ion on a sponge carrier.

2. The BMO@sponge biomaterial of claim 1, wherein the manganese oxidizing bacterium is MnB 1 deposited at American Type Culture Collection (ATCC) with an accession number of ATCC 23483.

3. A method for preparing the BMO@sponge biomaterial of claim 1, comprising:
preparing and sterilizing a culture medium;
sequentially adding a divalent manganese ion stock solution and the sponge carrier to the culture medium; and
inoculating the manganese oxidizing bacterium into the culture medium followed by incubation and freeze-drying to obtain the BMO@sponge biomaterial.

4. The method of claim 3, wherein the culture medium comprises 0.15 g/L ammonium ferrous sulfate, 0.075 g/L yeast leaching powder, 0.15 g/L sodium citrate, 0.05 g/L sodium pyrophosphate and 20 mM N-2-hydroxyethylpiperazine-N-2-ethanesulfonic acid, and has a pH of 7.0-7.2.

5. The method of claim 3, wherein the divalent manganese ion stock solution is a MnSO₄·7H₂O solution, and the MnSO₄·7H₂O solution is added such that a final divalent manganese ion concentration in the culture medium is 5-20 mg/L.

6. The method of claim 3, wherein the sponge carrier has a size of 1×1×1 cm, and an addition amount of the sponge carrier is 20-40 blocks/L.

7. The method of claim 3, wherein the manganese oxidizing bacterium is cultured to a logarithmic phase with an optical density at 600 nm (OD₆₀₀) of 0.9-1.2, and inoculated into the culture medium at an inoculum amount of 1%-5% by volume of the culture medium.

8. A method for treating a wastewater polluted by an antibiotic, comprising:
adding the BMO@sponge biomaterial of claim 1 and peroxymonosulfate (PMS) to the wastewater to degrade the antibiotic.

9. The method of claim 8, wherein the antibiotic is selected from the group consisting of tetracycline, macrolide, aminoglycoside and a combination thereof.

10. The method of claim 8, wherein a dosage of the BMO@sponge biomaterial is 5-15 blocks/L with each block having a size of 1 cm×1 cm×1 cm; a dosage of the PMS is 50-400 mg/L; and the wastewater has a pH of 7-9.
